(19) United States
(12) Patent Application Publication
(10) Pub. No.: US 2008/0045603 A1
(43) Pub. Date: Feb. 21, 2008

(54) Methods for treating at least one condition having MT1 receptor, 5HT2B receptor, and L-type calcium channel activity

(76) Inventors: John Devane, Co. Roscommon (IE); John Kelly, Dublin (IE); Mary Martin, Dublin (IE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/826,804

(22) Filed: Jul. 18, 2007

Related U.S. Application Data
(60) Provisional application No. 60/835,447, filed on Aug. 4, 2006.
(60) Provisional application No. 60/907,052, filed on Mar. 19, 2007.

(51) Int. Cl. A61K 31/135; A61P 1/00; A61P 11/00; A61P 11/06; A61P 25/00; A61P 35/00; A61P 9/12

(52) U.S. Cl. 514/653

(57) ABSTRACT

The present invention is directed to methods comprising administering a composition comprising a therapeutically effective amount of (R)-verapamil, a derivative thereof, or a pharmaceutically acceptable salt thereof, wherein the composition treats, prevents and/or manages at least one condition having MT1 receptor, 5-HT2B receptor and L-type calcium channel activity and releases the (R)-verapamil, a derivative thereof or a pharmaceutically acceptable salt thereof to exhibit a co-primary activity on the MT1 receptor, the 5-HT_{2B} receptor, and the L-type calcium channel.

This application claims priority to U.S. Provisional Patent Application No. 60/835,447, filed Aug. 4, 2006, and U.S. Provisional Patent Application No. 60/907,052, filed Mar. 19, 2007, both of which are incorporated herein by reference in their entirety.

The present invention is generally directed to methods for treating at least one condition having melatonin (MT1) receptor, 5-HT_{2B} receptor, and L-type calcium channel activity comprising administering a composition comprising a therapeutically effective amount of (R)-verapamil, a derivative thereof, or a pharmaceutically acceptable salt thereof, wherein the composition releases the (R)-verapamil, a derivative thereof or a pharmaceutically acceptable salt thereof to exhibit a co-primary activity on the MT1 receptor, the 5-HT_{2B} receptor, and the L-type calcium channel.

Generally, the goal of drug development focuses on compounds that are specific for a given receptor system and are selective for a given sub-receptor. Based on that specificity and selectivity, a compound's ideal use is a single mechanism of action. Using a single mechanism of action, the compound desirably produces very effective therapeutic effects without side effects from other mechanisms. However, single mechanism compounds may be associated with undesirable, potent side effects at tissues and on systems that are not implicated in treating the target disease. Those effects can result in serious side effects and/or adverse events.

For example, the 5-HT₃ antagonist Alosetron relieves the symptoms of irritable bowel syndrome (IBS), but has caused a number of patient deaths due to ischemic colitis. Another compound, Tegasorod, used to treat IBS, acts as a 5-HT₄ agonist. The U.S. FDA withdrew this compound from the market as well due to an increased incidence of myocardial infarctions. Likewise, the selective COX2 inhibitors used to treat inflammation conditions in lieu of COX1/2 inhibitors resulted in serious cardiovascular side effects.

However, in certain therapeutic areas, research suggests that there are therapeutic advantages observed by deliberately combining a number of distinct receptors and/or systems to achieve a superior therapeutic effect from a single compound. As such, drug development evolved from compounds with single action mechanisms to dual action mechanisms to multiple action mechanisms, i.e., activity at a number of receptors and/or systems. For instance, to be able to combine calcium channel activity, 5-HT_{2B} receptor activity, and melatonin receptor activity in a single compound, such a compound can have particular utility in treating, preventing and/or managing a wide range of seemingly unrelated diseases and/conditions.

Calcium channel (L-gated) blockers have established utility in cardiovascular disease. Calcium channels are known to be iontropic, i.e., ion channels are controlled (opened and closed) by the binding of chemical messengers. For example, calcium channel blockers can be used to treat hypertension, angina and cardiac arrhythmias by reducing the calcium ion influx in cardiac muscle to reduce rate, contractility and oxygen requirements. Pharmaceutical agents with such blocker activity have limited use, if any, in non-cardiovascular areas such as gastrointestinal conditions, e.g., irritable bowel syndrome.

On the other hand, 5-HT receptors are known to be metabotropic, i.e., a surface receptor that is not in the form of an ion channel but instead is indirectly linked with ion channels through signal transduction mechanisms. 5-HT_{2B} antagonists have been proposed as having utility in gastrointestinal conditions (e.g., gastrointestinal hypermotility), dyspepsia including functional dyspepsia, and gastro-esophageal reflux disease. Research also suggests that 5-HT_{2B} antagonists benefit conditions such as migraines, cluster headaches, cyclic vomiting, primary pulmonary disease, pulmonary arterial hypertension (PAH), restenosis, asthma, chronic obstructive pulmonary disease, prostyatic hyperplasia, generalized anxiety disorder, panic disorders, obsessive compulsive disorders, alcoholism, depression, sleep disorders and anorexia nervosa. Although 5-HT_{2B} antagonists potentially can be used to treat a variety of diseases and/or conditions, their use has been limited to cardiovascular agents, such as blood pressure agents.

Like 5-HT receptors, melatonin receptors are also metabotropic. Melatonin is a natural hormone that regulates circadian rhythms and seasonal responses to light-dark cycles and binds to sub-receptors such as MT1 and MT2. While melatonin is primarily produced in the pineal gland, the gut is also a major production source. It has been proposed that melatonin regulates gut motility and may have opposite effects to 5-HT, which is a precursor hormone and has been implicated in visceral hypersensitivity such as occurs in IBS.

A number of clinical studies also suggest that melatonin plays a therapeutic role in relieving pain in IBS. However, melatonin has activity throughout the body and is active in the central nervous system (CNS), the cardiovascular system, the kidneys, particular cells such as immune cells and adipocytes cells, reproductive function, and skin. In fact, melatonin is equiactive on the MT1 and MT2 sub-receptors, which explains, among other things, the variety of effects attributed to melatonin, and demonstrates that melatonin is nonselective. Other nonselective compounds for the MT1 and MT2 sub-receptors include an MT1/MT2 agonist that was recently approved in the USA for the treatment of insomnia (i.e., Ramelteon). In addition, a combined MT1/MT2 agonist and 5-HT_{2C} antagonist (i.e., Agomelatine) is in Phase III development for depression.

To be able to combine the activity from the calcium channel blockers with the 5-HT_{2B} antagonists, and MT1 agents can have particular utility in treating, preventing and/or managing a wide range of seemingly unrelated diseases and/or conditions. Thus, there is a need in the art for compositions and methods for treating, preventing, and/or managing diseases and/or conditions associated with the activity of calcium channels, and MT1 and 5-HT_{2B} receptors, i.e., having triple action.

For example, verapamil (benzeneacetonitrile .alpha.-[3-[[2-(3,4-dimethoxyphenyl)ethyl]methylamino]propyl]-3,4-dimethoxy-.alpha.-(1-methylethyl) hydrochloride) is a commercially available drug that, when used to treat cardiovascular conditions, acts as a calcium ion influx inhibitor by blocking calcium ion channels. This drug is typically prescribed as a treatment for cardiovascular conditions, such as hypertension, arterial fibrillation, angina, and paroxysmal supraventricular tachycardia. The drug is normally prescribed as a racemic mixture containing approximately equal amounts of (R)-verapamil and (S)-verapamil.

The pharmacodynamics and pharmacokinetics of the (R)- and (S)-stereoisomers, however, differ. For example, the (S)-isomer is typically 10 times more potent, i.e., effective, than the (R)-isomer at treating cardiovascular conditions. In addition, following oral administration of the racemate, stereo-selective first pass liver metabolism occurs that results in higher systemic concentrations (i.e., bioavailability) of the (R)-isomer. In addition, the inhibitory potency of the isomers against sites on the calcium channel and alpha-1-adrenergic receptors is different (Piascik, Can. J. Physiol. Pharmacol., 68(3):439-446, 1990).

Verapamil also causes several undesirable dose-limiting side effects. These include, inter alia, depression in myocardial activity (Satoh et al., J. Cardio. Pharm., 2: 309-318, 1980) and constipation (Hedner et al., Acta Pharmacol. Toxicol., 58 (Suppl 2): 119-30,1986; Krevsky et al., Dig. Dis. Sci., 37 (6): 919-924, 1992; Thulin, et al., Scand. J. Prim. Health Care Suppl., 1: 81-84, 1990). Researchers have attempted to overcome these unwanted side effects by using the individual stereoisomers of verapamil. Harding et al. (U.S. Pat. No. 5,889,060) describe the use of a single stereoisomer, (R)-verapamil, as a treatment for angina. Others suggest that (S)-verapamil is more beneficial for treating angina and arterial fibrillation, while (R)-verapamil is useful for reversing multi-drug resistance in cancer chemotherapy (e.g., McCague et al., U.S. Pat. No. 5,910,601; Harding et al., U.S. Pat. No. 5,932,246).

Longstreth et al. (U.S. Pat. No. 5,955,500) report that the ratio of (R)- and (S)-verapamil may be manipulated to achieve desirable cardiovascular effects while minimizing adverse effects such as slowing of cardiac conduction, alteration in heart rate, and constipation. Such a strategy has led to the development of a dosage form that releases the stereoisomers of verapamil at different rates in the body for the treatment of cardiovascular conditions (Gilbert et al., U.S. Pat. No. 6,267,980).

Harding et al. (U.S. Pat. No. 5,932,246) report that the separate administration of either (R)- or (S)-verapamil reduces the significant constipative effects caused by racemic verapamil. The patentees suggest that this therapeutic approach may achieve the desirable cardiovascular effects of verapamil, while reducing the constipation experienced by a patient undergoing treatment.

In contrast, other researchers have attempted to use the constipative effects of racemic verapamil as means for treating intestinal conditions (see, e.g., McCleod, Med. J. Aust., 2(3):119 (letter), 1983). Byrne (J. Clin. Psy., 48: 9, 1987) describes the treatment of 3 patients diagnosed with irritable bowel syndrome, and reports that 80 mg of racemic verapamil had a constipating effect on the patients. Similarly, Ahlman et al. (Br. J. Cancer, 54: 251-256, 1986) describe the treatment of a patient suffering from mid-gut carcinoid syndrome (experiencing severe bouts of diarrhea). According to Ahlman, low doses of racemic verapamil relieved the diarrhea.

Racemic verapamil or (S)-verapamil show calcium channel binding affinity, as when used to treat cardiovascular conditions, but they also cause several undesirable side-effects. On the other hand, enriched (R)-verapamil has been shown to exhibit intestinal selectivity, as provided in U.S. Pat. No. 6,849,661 to Kelly et al. It, however, has now been surprisingly discovered that the intestinal selectivity may be the result of selective 5-HT_{2B} receptor activity in relation to other 5-HT sub-receptors and transporter and that this 5-HT_{2B} receptor selectivity is similar in potency to its binding to calcium channels (L-gated) and MT1 receptors. Based on this selectivity, (R)-verapamil combines the calcium channel blocker activity with the 5-HT_{2B} antagonist activity as well as MT1 activity, i.e., triple action. In contrast, racemic and (S)-verapamil exhibit selective calcium channel binding but are non-selective for 5-HT receptor sub-types and transporter.

E. O. Okoro in 51 J. Pharm. Pharmacol. 953-57 (1999) suggests a link in the pharmacology of L-type calcium channel blockers and 5-HT₂ receptor antagonists. These findings, however, were in rat aorta tissue and do not make distinctions between the various components of the sub-receptors comprising the 5HT₂ receptor system. In addition, the findings are based in part on verapamil without delineating between the isomers of verapamil.

While the above-cited reports and others have described (R)-verapamil's use in treating some intestinal and cardiovascular conditions, none of those reports has sought to identity or characterize the selectivity of (R)-verapamil or utilize that combined selectivity to treat specific conditions. For example, the present inventors identified particular methods that result in effective therapy and improved safety by combining activity of distinct receptors, i.e., combining activity on the MT1 receptor, the 5-HT_{2B} receptor, and L-type calcium channel. Thus, an L-type calcium channel agent may be considered as an option to relieve, e.g., a migraine. Likewise, individual 5-HT_{2B} antagonists may also be considered to treat, e.g., migraines. Melatonin (the third receptor) has already been implicated in migraine treatment. As such, the present invention achieves a superior treatment for various conditions and/or diseases such as migraine by combining those different activities, while minimizing non-selective effects.

Accordingly, the present invention is directed to methods for treating, preventing, and/or managing at least one condition having 5-HT_{2B} receptor activity, MT1 receptor activity and L-type calcium channel activity comprising administering a therapeutically effective amount of (R)-verapamil, a derivative thereof, or pharmaceutically acceptable salt thereof, wherein the composition releases the (R)-verapamil, a derivative thereof or a pharmaceutically acceptable salt thereof to exhibit a co-primary activity on the MT1 receptor, the 5-HT_{2B} receptor, and the L-type calcium channel.

Because the therapeutic benefits can be described as the summation of three distinct mechanisms, the desired effect can be achieved minimizing the risk of affecting the functionality of the target receptors at tissues and organs that are not involved in, e.g., migraines and mitigate such side effects. The present invention can exploit co-primary pharmacology and at the same time, can be selective.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

As used herein, the term “selectivity” may be in terms of IC₅₀ binding activity (50% inhibitory concentration), EC₅₀ activity (50% effective concentration), or any other known selectivity parameter known to a person of ordinary skill in the art demonstrating selective effects on the 5-HT_{2B} and MT₁ receptors and calcium channels (L-gated) activity. The term “(R)-verapamil” encompasses a composition having a greater amount of the (R)-enantiomer or stereoisomer of verapamil than (S)-verapamil, derivatives and analogs thereof, and pharmaceutically acceptable salts thereof.

As used herein, the phrase “modified-release” formulation or dosage form includes a pharmaceutical preparation that achieves a desired release of the drug from the formulation. For example, a modified-release formulation may extend the influence or effect of a therapeutically effective dose of a pharmaceutically active compound in a patient. Such formulations are referred to herein as “extended-release formulations.” In addition to maintaining therapeutic levels of the pharmaceutically active compound, a modified-release formulation may also be designed to delay the release of the active compound for a specified period. Such compounds are referred to herein as “delayed onset” of “delayed release” formulations or dosage forms. Still further, modified-release formulations may exhibit properties of both delayed and extended release formulations, and thus be referred to as “delayed-onset, extended-release” formulations.

As used herein, the term “pharmaceutically acceptable excipient” includes compounds that are compatible with the other ingredients in a pharmaceutical formulation and not injurious to the subject when administered in therapeutically effective amounts.

As used herein, the term “pharmaceutically acceptable salt” includes salts that are physiologically tolerated by a subject. Such salts are typically prepared from an inorganic and/or organic acid. Examples of suitable inorganic acids include, but are not limited to, hydrochloric, hydrobromic, hydroiodic, nitric, sulfuric, and phosphoric acid. Organic acids may be aliphatic, aromatic, carboxylic, and/or sulfonic acids. Suitable organic acids include, but are not limited to, formic, acetic, propionic, succinic, camphorsulfonic, citric, fumaric, gluconic, lactic, malic, mucic, tartaric, para-toluenesulfonic, glycolic, glucuronic, maleic, furoic, glutamic, benzoic, anthranilic, salicylic, phenylacetic, mandelic, pamoic, methanesulfonic, ethanesulfonic, pantothenic, benzenesulfonic (besylate), stearic, sulfanilic, alginic, galacturonic, and the like.

The term “racemic” as used herein means a mixture of the enantiomers, or stereoisomers, of verapamil, a derivative thereof or a pharmaceutically acceptable salt thereof in which neither enantiomer, or stereoisomer, is substantially purified from the other.

The phrase “therapeutically effective amount of (R)-verapamil,” as used herein, refers to the amount of (R)-verapamil (a derivative thereof or a pharmaceutically acceptable salt thereof), which alone or in combination with other drugs, provides any therapeutic benefit in the prevention, treatment, and/or management of diseases and/or conditions associated with the activity of MT1, 5-HT_{2B} receptors, and calcium channels.

The term “antagonist,” as used herein, refers to agents or drugs that neutralize or impede the action or effects of others, e.g., a drug that binds to a receptor without eliciting a biological response and effectively blocking the binding of a substance that could elicit such a response. Antagonists may be competitive and reversible by reversibly binding to a region of a receptor in common with the agonist or competitive and irreversible by covalently binding to the agonist binding site. Antagonists may also be non-competitive where the antagonist binds to an allosteric site on the receptor or an associated ion channel.

As used herein, the term “co-primary activity” and/or “co-primary pharmacology” includes at least an agent that interacts with more than one receptor and/or system for activating or inhibiting normal body processes. For example, the composition of the present invention can release the (R)-verapamil, a derivative thereof or a pharmaceutically acceptable salt thereof to exhibit at least five times more activity on the 5-HT_{2B} receptor compared with other 5-HT receptors; release the (R)-verapamil, a derivative thereof or a pharmaceutically acceptable salt thereof to exhibit at least five times more activity on the MT1 receptor compared with the MT2 receptor; release the (R)-verapamil, a derivative thereof or a pharmaceutically acceptable salt thereof to exhibit a binding activity on the L-type calcium channel and at least equi-active binding activity on the 5-HT_{2B} and MT1 receptors; or release the (R)-verapamil, a derivative thereof or a pharmaceutically acceptable salt thereof to exhibit a ratio of calcium channel:5-HT_{2B}:MT1 binding activity of 1:at least 1 at least 1.

The invention is directed to methods for treating, preventing, and/or managing diseases and/or conditions associated with the activity of MT1 and 5-HT_{2B} receptors, and calcium channels comprising administering a therapeutically effective amount of (R)-verapamil, a derivative thereof, or a pharmaceutically acceptable salt thereof.

By combining co-primary pharmacology, selectivity and given the distinct characteristics of the target receptors (metabotropic and iontropic), the target therapeutic indications (i.e., diseases and/or conditions) include a number of overlapping conditions that at least implicate calcium flux, 5HT, and melatonin. Such diseases and/or conditions being treated, prevented and/or managed by the present invention are chosen from non GI-motility linked or secretory linked gastrointestinal conditions (i.e., excluding GI-motility linked gastrointestinal conditions), migraine headaches, cluster headaches, cyclic vomiting, increased intraocular pressure including glaucoma, primary pulmonary hypertension, restenosis, asthma, chronic obstructive pulmonary disease (COPD), prostatic hyperplasia, generalized anxiety disorder (GAD), panic disorders, obsessive compulsive disorders (OCD), alcoholism, depression, sleep disorders, anorexia nervosa, and diseases and/or conditions thereof. For example, the non GI-motility linked or secretory linked gastrointestinal conditions include, but not limited to, dyspepsia, functional dyspepsia, gastro-esophageal reflux disease, and diseases and/or conditions thereof. In addition, such diseases and/or conditions may also include, for example, but are not limited to, diarrhea-related or linked symptoms, chronic diarrhea, cancer-related diarrhea (e.g., colon cancer), carcinoid syndrome, chemotherapy and radiotherapy linked diarrhea, AIDS related diarrhea, food intolerance and malabsorption related diarrhea, medicine linked diarrhea including antibiotics, celiac disease, and endocrine diseases such as Addisons disease related diarrhea.

In at least one embodiment, (R)-verapamil, a derivative thereof or a pharmaceutically acceptable salt thereof, is provided in a composition for use in treating, preventing and/or managing diseases and/or conditions associated with the activity of MT1 and 5-HT_{2B} receptors, and calcium channels. Such compositions optionally comprise at least one pharmaceutically acceptable excipient. Suitable excipients are known to those of skill in the art and described, for example, in the Handbook of Pharmaceutical Excipients (Kibbe (ed.), 3rd Edition (2000), American Pharmaceutical Association, Washington, D.C.), and Remington's Pharmaceutical Sciences (Gennaro (ed.), 20th edition (2000), Mack Publishing, Inc., Easton, Pa.), which, for their disclosures relating to excipients and dosage forms, are incorporated herein by reference. For example, suitable excipients include, but are not limited to, starches, sugars, microcrystalline cellulose, diluents, granulating agents, lubricants, binders, disintegrating agents, wetting agents, emulsifiers, coloring agents, release agents, coating agents, sweetening agents, flavoring agents, perfuming agents, preservatives, plasticizers, gelling agents, thickeners, hardeners, setting agents, suspending agents, surfactants, humectants, carriers, stabilizers, antioxidants, and combinations thereof.

The pharmaceutical compositions of the invention are typically provided in dosage forms that are suitable for administration to a subject by a desired route. A number of suitable dosage forms are described below, but are not meant to include all possible choices. One of skill in the art is familiar with the various dosage forms that are suitable for use in the present invention, as described, for example, in Remington's Pharmaceutical Sciences, which has been incorporated by reference above. The most suitable route in any given case will depend on the nature and severity of the disease and/or condition being prevented, treated, and/or managed. For example, the pharmaceutical compositions may be formulated for administration orally, nasally, rectally, intravaginally, parenterally, intracistemally, and topically including buccally and sublingually.

Formulations suitable for oral administration include, but are not limited to, capsules, cachets, pills, tablets, lozenges (using a flavored basis, usually sucrose and acacia or tragacanth), powders, granules, solutions, suspensions in an aqueous or non-aqueous liquid, oil-in-water or water-in-oil liquid emulsions, elixirs, syrups, pastilles (using an inert base, such as gelatin and glycerin, or sucrose and acacia), mouth washes, pastes, and the like; each containing a predetermined amount of (R)-verapamil, a derivative thereof or a pharmaceutically acceptable salt thereof to provide a therapeutic amount of the drug in one or more doses.

In solid dosage forms for oral administration (capsules, tablets, pills, powders, granules and the like), the (R)-verapamil, a derivative thereof or a pharmaceutically acceptable salt thereof is typically mixed with one or more pharmaceutically-acceptable excipients, including carriers, such as sodium citrate or dicalcium phosphate; fillers or extenders, such as starches, spray-dried or anhydrous lactose, sucrose, glucose, mannitol, dextrose, sorbitol, cellulose (e.g., microcrystalline cellulose; AVICEL™), dihydrated or anhydrous dibasic calcium phosphate, and/or silicic acid; binders, such as acacia, alginic acid, carboxymethylcellulose (sodium), cellulose (microcrystalline), dextrin, ethylcellulose, gelatin, glucose (liquid), guar gum, hydroxypropyl cellulose, hydroxypropyl methylcellulose, methylcellulose (e.g., methylcellulose 2910), polyethylene oxide, povidone, starch (pregelatinized) or syrup; humectants, such as glycerol; disintegrating agents, such as agar, calcium carbonate, potato or tapioca starch, alginic acid, certain silicates, pregelatinized starch, sodium starch glycolate (EXPLOTAB™), crosslinked providone, crosslinked sodium carboxymethylcellulose, clays, microcrystalline cellulose (e.g., AVICEL™), alginates, gums, and/or sodium carbonate; solution retarding agents, such as paraffin; absorption accelerators, such as quaternary ammonium compounds; wetting agents, such as cetyl alcohol or glycerol monostearate; absorbents, such as kaolin and bentonite clay; lubricants, such as talc, calcium stearate, magnesium stearate, solid polyethylene glycols, steric acid, sodium stearyl fumarate, magnesium lauryl sulfate, hydrogenated vegetable oil, and/or sodium lauryl sulfate; glidants, such as calcium silicate, magnesium silicate, colloidal anahydrous silica, and/or talc; flavoring agents, such as synthetic flavor oils and flavoring aromatics, natural oils, extracts from plant leaves, flowers, and fruits, including cinnamon oil, oil of wintergreen, peppermint oils, bay oil, anise oil, eucalyptus, thyme oil, vanilla, citrus oil (e.g., lemon, orange, grape, lime, and grapefruit), fruit essences (e.g., apple, banana, pear, peach, strawberry, raspberry, cherry, plum, pineapple, apricot, as so forth); coloring agents and/or pigments, such as titanium dioxide and/or dyes approved for use in food and pharmaceuticals; buffering agents; dispersing agents; preservatives; and/or diluents. The aforementioned excipients are given as examples only and are not meant to include all possible choices.

Any of these solid dosage forms may optionally be scored or prepared with coatings and shells, such as enteric coatings, and coatings for modifying the rate of release, examples of which are well known in the pharmaceutical-formulating art. For example, such coatings may comprise sodium carboxymethylcellulose, cellulose acetate, cellulose acetate phthalate, ethylcellulose, gelatin, pharmaceutical glaze, hydroxypropyl cellulose, hydroxypropyl methylcellulose, hydroxypropyl methyl cellulose phthalate, methacrylic acid copolymer, methylcellulose, polyethylene glycol, polyvinyl acetate phthalate, shellac, sucrose, titanium dioxide, wax, or zein. In one embodiment, the coating material comprises hydroxypropyl methylcellulose. The coating material may further comprise anti-adhesives, such as talc; plasticizers (depending on the type of coating material selected), such as castor oil, diacetylated monoglycerides, dibutyl sebacate, diethyl phthalate, glycerin, polyethylene glycol, propylene glycol, triacetin, triethyl citrate; opacifiers, such as titanium dioxide; and/or coloring agents and/or pigments. The coating process may be carried out by any suitable means, for example, by using a perforated pan system such as the GLATT™, ACCELACOTA™, and/or HICOATER™ apparatuses.

Tablets may be formed by any suitable process, which are known to those of ordinary skill in the art. For example, the ingredients may be dry-granulated or wet-granulated by mixing in a suitable apparatus before tabletting. Granules of the ingredients to be tabletted may also be prepared using suitable spray/fluidization or extrusion/spheronsation techniques.

With quick-release tablets, the choice of excipients generally allows a fast dissolution. The tablets may be conventional instant release tablets designed to be taken whole in the typical administration manner (i.e., with sufficient amount of water to facilitate swallowing). Alternatively the tablets may be formulated with suitable excipients to act as a fast dissolving and/or fast melting tablet in the oral cavity. Also, the tablet can be in the form of a chewable or effervescent dosage form. With effervescent dosage forms, the tablet is typically added to a suitable liquid that causes it to disintegrate, dissolve, and/or disperse.

Tablets typically are designed to have an appropriate hardness and friability to facilitate manufacture on an industrial scale using equipment to produce tablets at high speed. Also the tablets are usually packed or filled in all kinds of containers. If the tablet has an insufficient hardness or is friable, the tablet that is taken by the subject may be broken or crumbled into powder. As a consequence of this insufficient hardness or friability, the subject can no longer be certain that the amount of the dose is correct. It should be noted that the hardness of tablets, amongst other properties, is influenced by the shape of the tablets. Different shapes of tablets may be used according to the present invention. Tablets may be circular, oblate, oblong, or any other shape that is known in the art. The shape of the tablets may also influence the disintegration rate.

Any of the solid compositions may encapsulated in soft and hard gelatin capsules using any of the excipients described above. For example, the encapsulated dosage form may include fillers, such as lactose and microcrystalline; glidants, such as colloidal silicon dioxide and talc; lubricants, such as magnesium stearate; and disintegrating agents, such as starch (e.g., maize starch). Using capsule filling equipment, the ingredients to be encapsulated are milled together, sieved, mixed, packed together, and then delivered into a capsule. The lubricants may be present in an amount from about 0.5% (w/w) to about 2.0% (w/w). In one embodiment, the lubricant is about 1.25% (w/w) of the content of the capsule.

(R)-verapamil, a derivative thereof or a pharmaceutically acceptable salt thereof may also be formulated into a liquid dosage form for oral administration. Suitable formulations include emulsions, microemulsions, solutions, suspensions, syrups, and elixirs. These formulations optionally include diluents commonly used in the art, such as, for example, water or other solvents, solubilizing agents and emulsifiers, including, but not limited to, ethyl alcohol, isopropyl alcohol, ethyl carbonate, ethyl acetate, benzyl alcohol, benzyl benzoate, propylene glycol, 1,3-butylene glycol, oils, glycerol, tetrahydrofuryl alcohol, polyethylene glycols, fatty acid esters of sorbitan, and mixtures thereof. In addition, the liquid formulations optionally include adjuvants such as wetting agents, emulsifying and suspending agents, sweetening, flavoring, coloring, perfuming and preservative agents. Suitable suspension agents include, but are not limited to, ethoxylated isostearyl alcohols, polyoxyethylene sorbitol and sorbitan esters, microcrystalline cellulose, aluminum metahydroxide, bentonite, agar-agar and tragacanth, xanthan gum, hydroxypropylmethylcellulose, methylcellulose, carageenan, sodium carboxymethyl cellulose, and sodium carboxymethyl cellulose/microcrystalline cellulose mixtures, sodium carboxymethyl cellulose/microcrystalline cellulose mixtures, and/or mixtures thereof. In one embodiment, the suspending agent comprises xanthan gum, carageenan, sodium carboxymethyl cellulose/microcrystalline cellulose mixtures, and/or mixtures thereof. In another embodiment, the suspending agent is AVICEL™ RC591, AVICEL™ RC581, and/or AVICEL™ CL611 (Avicel is a trademark of FMC Corporation); and/or RC591, RC581 and CL611 (mixtures of microcrystalline cellulose and sodium carboxymethyl cellulose).

The amount of suspending agent present will vary according to the particular suspending agent used and the presence or absence of other ingredients, which have an ability to act as a suspending agent or contribute significantly to the viscosity of the composition. The suspension may also contain ingredients to improve its taste, for example sweeteners; bitter-taste maskers, such as sodium chloride; taste-masking flavors, such as contramarum; flavor enhancers, such as monosodium glutamate; and flavoring agents. Examples of sweeteners include bulk sweeteners, such as sucrose, hydrogenated glucose syrup, the sugar alcohols sorbitol and xylitol; and sweetening agents such as sodium cyclamate, sodium saccharin, aspartame, and ammonium glycyrrhizinate. The liquid formulations may further comprise at least one buffering agent, as needed, to maintain the desired pH.

The liquid formulations of the present invention may also be filled into soft gelatin capsules. For example, the liquid may include a solution, suspension, emulsion, microemulsion, precipitate, or any other desired liquid media carrying (R)-verapamil, a derivative thereof or a pharmaceutically acceptable salt thereof. The liquid may be designed to improve the solubility of (R)-verapamil, a derivative thereof or a pharmaceutically acceptable salt thereof upon release, or may be designed to form a drug-containing emulsion or dispersed phase upon release. Examples of such techniques are well known in the art. Soft gelatin capsules may be coated, as desired, with a functional coating, as described below, to delay the release of the drug.

For rectal or vaginal administration, the composition may be provided as a suppository. Suppositories optionally comprise at least one non-irritating excipient, for example, polyethylene glycol, a suppository wax, or a salicylate. Such excipients may be selected on the basis of desirable physical properties. For example, a compound that is solid at room temperature but liquid at body temperature will melt in the rectum or vaginal cavity and release the active compound. The formulation may alternatively be provided as an enema for rectal delivery. Formulations suitable for vaginal administration also include pessaries, tampons, creams, gels, pastes, foams or spray formulations containing such carriers, examples of which are known in the art.

Formulations suitable for topical or transdermal administration include powders, sprays, ointments, pastes, creams, lotions, gels, solutions, patches and inhalants. Such formulations optionally contain excipients such as animal and vegetable fats, oils, waxes, paraffins, starch, tragacanth, cellulose derivatives, polyethylene glycols, silicones, bentonites, silicic acid, talc, zinc oxide, or mixtures thereof. Powders and sprays may also contain excipients such as lactose, talc, silicic acid, aluminum hydroxide, calcium silicates and polyamide powder. Additionally, sprays may contain propellants, such as chlorofluoro-hydrocarbons and volatile unsubstituted hydrocarbons, such as butane and propane.

Transdermal patches have the added advantage of providing controlled delivery of the mixture of the invention to the body. Such dosage forms can be made by dissolving, dispersing or otherwise incorporating a pharmaceutical composition containing (R)-verapamil, a derivative thereof or a pharmaceutically acceptable salt thereof in a suitable medium, such as an elastomeric matrix material. Absorption enhancers can also be used to increase the flux of the mixture across the skin. The rate of such flux can be controlled by either providing a rate-controlling membrane or dispersing the compound in a polymer matrix or gel.

For parenteral administration, such as administration by injection (including, but not limited to, subcutaneous, bolus injection, intramuscular, intraperitoneal, and intravenous), the pharmaceutical compositions may be formulated as isotonic suspensions, solutions or emulsions in oily or aqueous vehicles, and may contain formulatory agents such as suspending, stabilizing, or dispersing agents. Alternatively, the compositions may be provided in dry form such as a powder, crystalline or freeze-dried solid for reconstitution with sterile pyrogen-free water or isotonic saline before use. They may be presented, for example, in sterile ampoules or vials.

Examples of suitable aqueous and nonaqueous excipients include water, ethanol, polyols (such as glycerol, propylene glycol, polyethylene glycol, and the like), methacrylate), poly(ethyl methacrylate), poly(butyl methacrylate), poly(isobutyl methacrylate), and poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), poly(ethylene), poly(ethylene) low density, poly(ethylene) high density, poly(ethylene oxide), poly(ethylene terephthalate), poly(vinyl isobutyl ether), poly(vinyl acetate), poly(vinyl chloride) or polyurethane, and/or mixtures thereof.

Suitable pharmaceutically acceptable excipients include, but are not limited to, carriers, such as sodium citrate and dicalcium phosphate; fillers or extenders, such as stearates, silicas, gypsum, starches, lactose, sucrose, glucose, mannitol, talc, and silicic acid; binders, such as hydroxypropyl methylcellulose, hydroxymethyl cellulose, alginates, gelatin, polyvinyl pyrrolidone, sucrose and acacia; humectants, such as glycerol; disintegrating agents, such as agar, calcium carbonate, potato and tapioca starch, alginic acid, certain silicates, EXPLOTAB™, crospovidone, and sodium carbonate; solution retarding agents, such as paraffin; absorption accelerators, such as quaternary ammonium compounds; wetting agents, such as cetyl alcohol and glycerol monostearate; absorbents, such as kaolin and bentonite clay; lubricants, such as talc, calcium stearate, magnesium stearate, solid polyethylene glycols, and sodium lauryl sulfate; stabilizers, such as fumaric acid; coloring agents; buffering agents; dispersing agents; preservatives; organic acids; and organic bases. The aforementioned excipients are given as examples only and are not meant to include all possible choices. Additionally, many excipients may have more than one role or function, or be classified oils, injectable organic esters, and mixtures thereof. Proper fluidity can be maintained, for example, by the use of coating materials and surfactants.

These compositions may also contain adjuvants such as preservatives, wetting agents, emulsifying agents, and dispersing agents. Prevention of the action of microorganisms may be achieved by the inclusion of various antibacterial and/or antifungal agents, for example, paraben, chlorobutanol, phenol sorbic acid, and the like. It may also be desirable to include isotonic agents, such as sugars, sodium chloride, and the like in the compositions. In addition, prolonged absorption of the injectable pharmaceutical form may be brought about by the inclusion of agents which delay absorption such as aluminum monostearate and gelatin.

In order to prolong the therapeutic effect of a drug, it is often desirable to slow the absorption of the drug from a subcutaneous or intramuscular injection. This may be accomplished by the use of a liquid suspension of crystalline or amorphous material having low solubility. The rate of absorption of the drug then depends upon its rate of dissolution which, in turn, may depend upon crystal size and crystalline form. Alternatively, delayed absorption of a parenterally-administered form can be accomplished by dissolving or suspending the drug in an oil vehicle.

In addition to the common dosage forms described above, the compositions of the present invention may be formulated into a dosage form that modifies the release of (R)-verapamil, a derivative thereof or a pharmaceutically acceptable salt thereof. Examples of suitable modified release formulations, which may be used in accordance with the present invention include, but are not limited to, matrix systems, osmotic pumps, and membrane controlled dosage forms. These formulations typically comprise (R)-verapamil, a derivative thereof or a pharmaceutically acceptable salt thereof. Suitable pharmaceutically acceptable salts are discussed above.

Different types of modified dosage forms are briefly described below. A more detailed discussion of such forms may also be found in, for example The Handbook of Pharmaceutical Controlled Release Technology, D. L. Wise (ed.), Marcel Dekker, Inc., New York (2000); and also in Treatise on Controlled Drug Delivery: Fundamentals, Optimization, and Applications, A. Kydonieus (ed.), Marcel Dekker, Inc., New York, (1992), the relevant contents of each of which is hereby incorporated by reference for this purpose. Examples of modified release dosage forms are also described, for example, in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,566, the disclosures of which, for their discussions of pharmaceutical formulations, are incorporated herein by reference.

Advantages of modified-release formulations may include extended activity of the drug, reduced dosage frequency, increased patient compliance, and the ability to deliver the drug to specific sites in the intestinal tract. Suitable components (e.g., polymers, excipients, etc.) for use in modified-release formulations, and methods of producing the same, are also described, e.g., in U.S. Pat. No. 4,863,742, which is incorporated by reference for these purposes.

Matrix-Based Dosage Forms

In some embodiments, the modified release formulations of the present invention are provided as matrix-based dosage forms. Matrix formulations according to the invention may include hydrophilic, e.g., water-soluble, and/or hydrophobic, e.g., water-insoluble, polymers. The matrix formulations of the present invention may optionally be prepared with functional coatings, which may be enteric, e.g., exhibiting a pH-dependent solubility, or non-enteric, e.g., exhibiting a pH-independent solubility.

Matrix formulations of the present invention may be prepared by using, for example, direct compression or wet granulation. A functional coating, as noted above, may then be applied in accordance with the invention. Additionally, a barrier or sealant coat may be applied over a matrix tablet core prior to application of a functional coating. The barrier or sealant coat may serve the purpose of separating an active ingredient from a functional coating, which may interact with the active ingredient, or it may prevent moisture from contacting the active ingredient. Details of barriers and sealants are provided below.

In a matrix-based dosage form in accordance with the present invention, (R)-verapamil and optional pharmaceutically acceptable excipient(s) are dispersed within a polymeric matrix, which typically comprises at least one water-soluble polymer and/or at least one water-insoluble polymer. The drug may be released from the dosage form by diffusion and/or erosion. Such matrix systems are described in detail by Wise and Kydonieus, supra.

Suitable water-soluble polymers include, but are not limited to, polyvinyl alcohol, polyvinylpyrrolidone, methylcellulose, hydroxypropylcellulose, hydroxypropylmethyl cellulose or polyethylene glycol, and/or mixtures thereof.

Suitable water-insoluble polymers include, but are not limited to, ethylcellulose, cellulose acetate cellulose propionate, cellulose acetate propionate, cellulose acetate butyrate, cellulose acetate phthalate, cellulose triacetate, poly(methyl in more than one group; the classifications are descriptive only, and not intended to limit any use of a particular excipient.

In one embodiment, a matrix-based dosage form comprises (R)-verapamil; a filler, such as starch, lactose, or microcrystalline cellulose (AVICEL™); a binder/controlled-release polymer, such as hydroxypropyl methylcellulose or polyvinyl pyrrolidone; a disintegrant, such as, EXPLOTAB™, crospovidone, or starch; a lubricant, such as magnesium stearate or stearic acid; a surfactant, such as sodium lauryl sulfate or polysorbates; and a glidant, such as colloidal silicon dioxide (AEROSIL™) or talc.

The amounts and types of polymers, and the ratio of water-soluble polymers to water-insoluble polymers in the inventive formulations are generally selected to achieve a desired release profile of (R)-verapamil, a derivative thereof or a pharmaceutically acceptable salt thereof. For example, by increasing the amount of water-insoluble-polymer relative to the amount of water-soluble polymer, the release of the drug may be delayed or slowed. This is due, in part, to an increased impermeability of the polymeric matrix, and, in some cases, to a decreased rate of erosion during transit through the GI tract.

Osmotic Pump Dosage Forms

In another embodiment, the modified release formulations of the present invention are provided as osmotic pump dosage forms. In an osmotic pump dosage form, a core containing (R)-verapamil, a derivative thereof or a pharmaceutically acceptable salt thereof, and optionally at least one osmotic excipient is typically encased by a selectively permeable membrane having at least one pore or orifice. The selectively permeable membrane is generally permeable to water, but impermeable to the drug. When the system is exposed to body fluids, water penetrates through the selectively permeable membrane into the core containing the drug and optional osmotic excipients. The osmotic pressure increases within the dosage form. Consequently, the drug is released through the pores or orifice(s) in an attempt to equalize the osmotic pressure across the selectively permeable membrane.

In more complex pumps, the dosage form may contain two internal compartments in the core. The first compartment contains the drug and the second compartment may contain a polymer, which swells on contact with aqueous fluid. After ingestion, this polymer swells into the drug-containing compartment, diminishing the volume occupied by the drug, thereby delivering the drug from the device at a controlled rate over an extended period of time. Such dosage forms are often used when a zero order release profile is desired.

Osmotic pumps are well known in the art. For example, U.S. Pat. Nos. 4,088,864, 4,200,098, and 5,573,776, each of which is hereby incorporated by reference for this purpose, describe osmotic pumps and methods of their manufacture. The osmotic pumps useful in accordance with the present invention may be formed by compressing a tablet of an osmotically active drug, or an osmotically inactive drug in combination with an osmotically active agent, and then coating the tablet with a selectively permeable membrane, which is permeable to an exterior aqueous-based fluid but impermeable to the drug and/or osmotic agent.

At least one delivery orifice may be drilled through the selectively permeable membrane wall. Alternatively, at least one orifice in the wall may be formed by incorporating leachable pore-forming materials in the wall. In operation, the exterior aqueous-based fluid is imbibed through the selectively permeable membrane wall and contacts the drug to form a solution or suspension of the drug. The drug solution or suspension is then pumped out through the orifice as fresh fluid is imbibed through the selectively permeable membrane.

Typical materials for the selectively permeable membrane include selectively permeable polymers known in the art to be useful in osmosis and reverse osmosis membranes, such as cellulose acylate, cellulose diacylate, cellulose triacylate, cellulose acetate, cellulose diacetate, cellulose triacetate, agar acetate, amylose triacetate, beta glucan acetate, acetaldehyde dimethyl acetate, cellulose acetate ethyl carbamate, polyamides, polyurethanes, sulfonated polystyrenes, cellulose acetate phthalate, cellulose acetate methyl carbamate, cellulose acetate succinate, cellulose acetate dimethyl aminoacetate, cellulose acetate ethyl carbamate, cellulose acetate chloracetate, cellulose dipalmitate, cellulose dioctanoate, cellulose dicaprylate, cellulose dipentanlate, cellulose acetate valerate, cellulose acetate succinate, cellulose propionate succinate, methyl cellulose, cellulose acetate p-toluene sulfonate, cellulose acetate butyrate, lightly cross-linked polystyrene derivatives, cross-linked poly(sodium styrene sulfonate), poly(vinylbenzyltrimethyl ammonium chloride), cellulose acetate, cellulose diacetate, cellulose triacetate, and/or mixtures thereof.

The osmotic agents that can be used in the pump are typically soluble in the fluid that enters the device following administration, resulting in an osmotic pressure gradient across the selectively permeable wall against the exterior fluid. Suitable osmotic agents include, but are not limited to, magnesium sulfate, calcium sulfate, magnesium chloride, sodium chloride, lithium chloride, potassium sulfate, sodium carbonate, sodium sulfite, lithium sulfate, potassium chloride, sodium sulfate, d-mannitol, urea, sorbitol, inositol, raffinose, sucrose, glucose, hydrophilic polymers such as cellulose polymers, and/or mixtures thereof.

As discussed above, the osmotic pump dosage form may contain a second compartment containing a swellable polymer. Suitable swellable polymers typically interact with water and/or aqueous biological fluids, which causes them to swell or expand to an equilibrium state. Acceptable polymers exhibit the ability to swell in water and/or aqueous biological fluids, retaining a significant portion of such imbibed fluids within their polymeric structure, so as into increase the hydrostatic pressure within the dosage form. The polymers may swell or expand to a very high degree, usually exhibiting a 2- to 50-fold volume increase. The polymers can be non-cross-linked or cross-linked. In one embodiment, the swellable polymers are hydrophilic polymers. Suitable polymers include, but are not limited to, poly(hydroxy alkyl methacrylate) having a molecular weight of from 30,000 to 5,000,000; kappa-carrageenan; polyvinylpyrrolidone having a molecular weight of from 10,000 to 360,000; anionic and cationic hydrogels; polyelectrolyte complexes; poly(vinyl alcohol) having low amounts of acetate, cross-linked with glyoxal, formaldehyde, or glutaraldehyde, and having a degree of polymerization from 200 to 30,000; a mixture including methyl cellulose, cross-linked agar and carboxymethyl cellulose; a water-insoluble, water-swellable copolymer produced by forming a dispersion of finely divided maleic anhydride with styrene, ethylene, propylene, butylene or isobutylene; water-swellable polymers of N-vinyl lactams; and/or mixtures of any of the foregoing.

The term “orifice” as used herein comprises means and methods suitable for releasing the drug from the dosage form. The expression includes one or more apertures or orifices that have been bored through the selectively permeable membrane by mechanical procedures. Alternatively, an orifice may be formed by incorporating an erodible element, such as a gelatin plug, in the selectively permeable membrane. In such cases, the pores of the selectively permeable membrane form a “passageway” for the passage of the drug. Such “passageway” formulations are described, for example, in U.S. Pat. Nos. 3,845,770 and 3,916,899, the relevant disclosures of which are incorporated herein by reference for this purpose.

The osmotic pumps useful in accordance with this invention may be manufactured by techniques known in the art. For example, the drug and other ingredients may be milled together and pressed into a solid having the desired dimensions (e.g., corresponding to the first compartment). The swellable polymer is then formed, placed in contact with the drug, and both are surrounded with the selectively permeable agent. If desired, the drug component and polymer component may be pressed together before applying the selectively permeable membrane. The selectively permeable membrane may be applied by any suitable method, for example, by molding, spraying, or dipping.

Membrane-Controlled Dosage Forms

The modified release formulations of the present invention may also be provided as membrane controlled formulations. Membrane controlled formulations of the present invention can be made by preparing a rapid release core, which may be a monolithic (e.g., tablet) or multi-unit (e.g., pellet) type, and coating the core with a membrane. The membrane-controlled core can then be further coated with a functional coating. In between the membrane-controlled core and functional coating, a barrier or sealant may be applied. Details of membrane-controlled dosage forms are provided below.

In one embodiment, (R)-verapamil, a derivative thereof or a pharmaceutically acceptable salt thereof may be provided in a multiparticulate membrane controlled formulation. (R)-verapamil, a derivative thereof or a pharmaceutically acceptable salt thereof may be formed into an active core by applying the drug to a nonpareil seed having an average diameter in the range of about 0.4 to about 1.1 mm or about 0.85 to about 1.00 mm. (R)-verapamil, a derivative thereof or a pharmaceutically acceptable salt thereof may be applied with or without additional excipients onto the inert cores, and may be sprayed from solution or suspension using a fluidized bed coater (e.g., Wurster coating) or pan coating system. Alternatively, the (R)-verapamil, a derivative thereof or a pharmaceutically acceptable salt thereof may be applied as a powder onto the inert cores using a binder to bind the (R)-verapamil onto the cores. Active cores may also be formed by extrusion of the core with suitable plasticizers (described below) and any other processing aids as necessary.

The modified release formulations of the present invention comprise at least one polymeric material, which may be applied as a membrane coating to the drug-containing cores. Suitable water-soluble polymers include, but are not limited to, polyvinyl alcohol, polyvinylpyrrolidone, methylcellulose, hydroxypropylcellulose, hydroxypropylmethyl cellulose or polyethylene glycol, and/or mixtures thereof.

Suitable water-insoluble polymers include, but are not limited to, ethylcellulose, cellulose acetate cellulose propionate, cellulose acetate propionate, cellulose acetate butyrate, cellulose acetate phthalate, cellulose triacetate, poly(methyl methacrylate), poly(ethyl methacrylate), poly(butyl methacrylate), poly(isobutyl methacrylate), and poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), poly(ethylene), poly(ethylene) low density, poly(ethylene) high density, poly(ethylene oxide), poly(ethylene terephthalate), poly(vinyl isobutyl ether), poly(vinyl acetate), poly(vinyl chloride) or polyurethane, and/or mixtures thereof.

EUDRAGIT™ polymers (available from Rohm Pharma) are polymeric lacquer substances based on acrylates and/or methacrylates. A suitable polymer that is freely permeable to the active ingredient and water is EUDRAGIT™ RL. A suitable polymer that is slightly permeable to the active ingredient and water is EUDRAGIT™ RS. Other suitable polymers which are slightly permeable to the active ingredient and water, and exhibit a pH-dependent permeability include, but are not limited to, EUDRAGIT™ L, EUDRAGIT™ S, and EUDRAGIT™ E.

EUDRAGIT™ RL and RS are acrylic resins comprising copolymers of acrylic and methacrylic acid esters with a low content of quaternary ammonium groups. The ammonium groups are present as salts and give rise to the permeability of the lacquer films. EUDRAGIT™ RL and RS are freely permeable (RL) and slightly permeable (RS), respectively, independent of pH. The polymers swell in water and digestive juices, in a pH-independent manner. In the swollen state, they are permeable to water and to dissolved active compounds.

EUDRAGIT™ L is an anionic polymer synthesized from methacrylic acid and methacrylic acid methyl ester. It is insoluble in acids and pure water. It becomes soluble in neutral to weakly alkaline conditions. The permeability of EUDRAGIT™ L is pH dependent. Above pH 5.0, the polymer becomes increasingly permeable.

In one embodiment comprising a membrane-controlled dosage form, the polymeric material comprises methacrylic acid co-polymers, ammonio methacrylate co-polymers, or a mixture thereof. Methacrylic acid co-polymers such as EUDRAGIT™ S and EUDRAGIT™ L (Rohm Pharma) are particularly suitable for use in the controlled release formulations of the present invention. These polymers are gastroresistant and enterosoluble polymers. Their polymer films are insoluble in pure water and diluted acids. They dissolve at higher pHs, depending on their content of carboxylic acid. EUDRAGIT™ S and EUDRAGIT™ L can be used as single components in the polymer coating or in combination in any ratio. By using a combination of the polymers, the polymeric material may exhibit a solubility at a pH between the pHs at which EUDRAGIT™ L and EUDRAGIT™ S are separately soluble.

The membrane coating may comprise a polymeric material comprising a major proportion (i.e., greater than 50% of the total polymeric content) of one or more pharmaceutically acceptable water-soluble polymers, and optionally a minor proportion (i.e., less than 50% of the total polymeric content) of one or more pharmaceutically acceptable water-insoluble polymers. Alternatively, the membrane coating may comprise a polymeric material comprising a major proportion (i.e., greater than 50% of the total polymeric content) of one or more pharmaceutically acceptable water-insoluble polymers, and optionally a minor proportion (i.e., less than 50% of the total polymeric content) of one or more pharmaceutically acceptable water-soluble polymers.

Ammonio methacrylate co-polymers such as Eudragit RS and Eudragit RL (Rohm Pharma) are suitable for use in the controlled release formulations of the present invention. These polymers are insoluble in pure water, dilute acids, buffer solutions, or digestive fluids over the entire physiological pH range. The polymers swell in water and digestive fluids independently of pH. In the swollen state they are then permeable to water and dissolved actives. The permeability of the polymers depends on the ratio of ethylacrylate (EA), methyl methacrylate (MMA), and trimethylammonioethyl methacrylate chloride (TAMCI) groups in the polymer. Those polymers having EA:MMA:TAMCI ratios of 1:2:0.2 (Eudragit RL) are more permeable than those with ratios of 1:2:0.1 (Eudragit RS). Polymers of Eudragit RL are insoluble polymers of high permeability. Polymers of Eudragit RS are insoluble films of low permeability.

The ammonio methacrylate co-polymers may be combined in any desired ratio. For example, a ratio of Eudragit RS:Eudragit RL (90:10) may be used. The ratios may furthermore be adjusted to provide a delay in release of the drug. For example, the ratio of Eudragit RS:Eudragit RL may be about 100:0 to about 80:20, about 100:0 to about 90:10, or any ratio in between. In such formulations, the less permeable polymer Eudragit RS would generally comprise the majority of the polymeric material.

The ammonio methacrylate co-polymers may be combined with the methacrylic acid co-polymers within the polymeric material in order to achieve the desired delay in release of the drug. Ratios of ammonio methacrylate co-polymer (e.g., Eudragit RS) to methacrylic acid co-polymer in the range of about 99:1 to about 20:80 may be used. The two types of polymers can also be combined into the same polymeric material, or provided as separate coats that are applied to the core.

In addition to the Eudragit polymers described above, a number of other such copolymers may be used to control drug release. These include methacrylate ester co-polymers (e.g., Eudragit NE 30D). Further information on the Eudragit polymers can be found in “Chemistry and Application Properties of Polymethacrylate Coating Systems,” in Aqueous Polymeric Coatings for Pharmaceutical Dosage Forms, ed. James McGinity, Marcel Dekker Inc., New York, pg 109-114.

The coating membrane may further comprise at least one soluble excipient so as to increase the permeability of the polymeric material. Suitably, the soluble excipient is selected from among a soluble polymer, a surfactant, an alkali metal salt, an organic acid, a sugar, and a sugar alcohol. Such soluble excipients include, but are not limited to, polyvinyl pyrrolidone, polyethylene glycol, sodium chloride, surfactants such as sodium lauryl sulfate and polysorbates, organic acids such as acetic acid, adipic acid, citric acid, fumaric acid, glutaric acid, malic acid, succinic acid, and tartaric acid, sugars such as dextrose, fructose, glucose, lactose and sucrose, sugar alcohols such as lactitol, maltitol, mannitol, sorbitol and xylitol, xanthan gum, dextrins, and maltodextrins. In some embodiments, polyvinyl pyrrolidone, mannitol, and/or polyethylene glycol can be used as soluble excipients. The soluble excipient(s) may be used in an amount of from about 1% to about 10% by weight, based on the total dry weight of the polymer.

In another embodiment, the polymeric material comprises at least one water-insoluble polymer, which are also insoluble in gastrointestinal fluids, and at lesat one water-soluble pore-forming compound. For example, the water-insoluble polymer may comprise a terpolymer of polyvinylchloride, polyvinylacetate, and/or polyvinylalcohol. Suitable water-soluble pore-forming compounds include, but are not limited to, saccharose, sodium chloride, potassium chloride, polyvinylpyrrolidone, and/or polyethyleneglycol. The pore-forming compounds may be uniformly or randomly distributed throughout the water-insoluble polymer. Typically, the pore-forming compounds comprise about 1 part to about 35 parts for each about 1 to about 10 parts of the water-insoluble polymers.

When such dosage forms come in to contact with the dissolution media (e.g., intestinal fluids), the pore-forming compounds within the polymeric material dissolve to produce a porous structure through which the drug diffuses. Such formulations are described in more detail in U.S. Pat. No. 4,557,925, which relevant part is incorporated herein by reference for this purpose. The porous membrane may also be coated with an enteric coating, as described herein, to inhibit release in the stomach.

In one embodiment, such pore forming controlled release dosage forms comprise (R)-verapamil, a derivative thereof or a pharmaceutically acceptable salt thereof; a filler, such as starch, lactose, or microcrystalline cellulose (AVICEL™); a binder/controlled release polymer, such as hydroxypropyl methylcellulose or polyvinyl pyrrolidone; a disintegrant, such as, EXPLOTAB™, crospovidone, or starch; a lubricant, such as magnesium stearate or stearic acid; a surfactant, such as sodium lauryl sulphate or polysorbates; and a glidant, such as colloidal silicon dioxide (AEROSIL™) or talc.

The polymeric material may also include one or more auxiliary agents such as fillers, plasticizers, and/or anti-foaming agents. Representative fillers include talc, fumed silica, glyceryl monostearate, magnesium stearate, calcium stearate, kaolin, colloidal silica, gypsum, micronized silica, and magnesium trisilicate. The quantity of filler used typically ranges from about 2% to about 300% by weight, and can range from about 20 to about 100%, based on the total dry weight of the polymer. In one embodiment, talc is the filler.

The coating membranes, and functional coatings as well, can also include a material that improves the processing of the polymers. Such materials are generally referred to as plasticizers and include, for example, adipates, azelates, benzoates, citrates, isoebucates, phthalates, sebacates, stearates and glycols. Representative plasticizers include acetylated monoglycerides, butyl phthalyl butyl glycolate, dibutyl tartrate, diethyl phthalate, dimethyl phthalate, ethyl phthalyl ethyl glycolate, glycerin, ethylene glycol, propylene glycol, triacetin citrate, triacetin, tripropinoin, diacetin, dibutyl phthalate, acetyl monoglyceride, polyethylene glycols, castor oil, triethyl citrate, polyhydric alcohols, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidised tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate, glyceryl monocaprylate, and glyceryl monocaprate. In one embodiment, the plasticizer is dibutyl sebacate. The amount of plasticizer used in the polymeric material typically ranges from about 10% to about 50%, for example, about 10%, 20%, 30%, 40%, or 50%, based on the weight of the dry polymer.

Anti-foaming agents can also be included. In one embodiment, the anti-foaming agent is simethicone. The amount of anti-foaming agent used typically comprises from about 0% to about 0.5% of the final formulation.

The amount of polymer to be used in the membrane controlled formulations is typically adjusted to achieve the desired drug delivery properties, including the amount of drug to be delivered, the rate and location of drug delivery, the time delay of drug release, and the size of the multiparticulates in the formulation. The amount of polymer applied typically provides an about 10% to about 100% weight gain to the cores. In one embodiment, the weight gain from the polymeric material ranges from about 25% to about 70%.

The combination of all solid components of the polymeric material, including co-polymers, fillers, plasticizers, and optional excipients and processing aids, typically provides an about 10% to about 450% weight gain on the cores. In one embodiment, the weight gain is about 30% to about 160%.

The polymeric material can be applied by any known method, for example, by spraying using a fluidized bed coater (e.g., Wurster coating) or pan coating system. Coated cores are typically dried or cured after application of the polymeric material. Curing means that the multiparticulates are held at a controlled temperature for a time sufficient to provide stable release rates. Curing can be performed, for example, in an oven or in a fluid bed drier. Curing can be carried out at any temperature above room temperature.

A sealant or barrier can also be applied to the polymeric coating. A sealant or barrier layer may also be applied to the core prior to applying the polymeric material. A sealant or barrier layer is not intended to modify the release of (R)-verapamil, a derivative thereof or a pharmaceutically acceptable salt. Suitable sealants or barriers are permeable or soluble agents such as hydroxypropyl methylcellulose, hydroxypropyl cellulose, hydroxypropyl ethylcellulose, and xanthan gum.

Other agents can be added to improve the processability of the sealant or barrier layer. Such agents include talc, colloidal silica, polyvinyl alcohol, titanium dioxide, micronized silica, fumed silica, glycerol monostearate, magnesium trisilicate and magnesium stearate, or a mixture thereof. The sealant or barrier layer can be applied from solution (e.g., aqueous) or suspension using any known means, such as a fluidized bed coater (e.g., Wurster coating) or pan coating system. Suitable sealants or barriers include, for example, OPADRY WHITE Y-1-7000 and OPADRY OY/B/28920 WHITE, each of which is available from Colorcon Limited, England.

The invention also provides an oral dosage form containing a multiparticulate (R)-verapamil, a derivative thereof or a pharmaceutically acceptable salt thereof, formulation as hereinabove defined, in the form of caplets, capsules, particles for suspension prior to dosing, sachets, or tablets. When the dosage form is in the form of tablets, the tablets may be disintegrating tablets, fast dissolving tablets, effervescent tablets, fast melt tablets, and/or mini-tablets. The dosage form can be of any shape suitable for oral administration of a drug, such as spheroidal, cube-shaped oval, or ellipsoidal. The dosage forms can be prepared from the multiparticulates in a manner known in the art and include additional pharmaceutically acceptable excipients, as desired.

All of the particular embodiments described above, including but not limited to, matrix-based, osmotic pump-based, soft gelatin capsules, and/or membrane-controlled forms, which may further take the form of monolithic and/or multi-unit dosage forms, may have a functional coating. Such coatings generally serve the purpose of delaying the release of the drug for a predetermined period. For example, such coatings may allow the dosage form to pass through the stomach without being subjected to stomach acid or digestive juices. Thus, such coatings may dissolve or erode upon reaching a desired point in the gastrointestinal tract, such as the upper intestine.

Such functional coatings may exhibit pH-dependent or pH-independent solubility profiles. Those with pH-independent profiles generally erode or dissolve away after a predetermined period, and the period is generally directly proportional to the thickness of the coating. Those with pH-dependent profiles, on the other hand, may maintain their integrity while in the acid pH of the stomach, but quickly erode or dissolve upon entering the more basic upper intestine.

Thus, a matrix-based, osmotic pump-based, or membrane-controlled formulation may be further coated with a functional coating that delays the release of the drug. For example, a membrane-controlled formulation may be coated with an enteric coating that delays the exposure of the membrane-controlled formulation until the upper intestine is reached. Upon leaving the acidic stomach and entering the more basic intestine, the enteric coating dissolves. The membrane-controlled formulation then is exposed to gastrointestinal fluid, and then releases (R)-verapamil, a derivative thereof or a pharmaceutically acceptable salt thereof over an extended period, in accordance with the invention. Examples of functional coatings such as these are well known to those in the art.

Any of the oral dosage forms described herein may be provided in the form of caplets, capsules, beads, granules, particles for suspension prior to dosing, sachets, or tablets. When the dosage form is in the form of tablets, the tablets may be disintegrating tablets, fast dissolving tablets, effervescent tablets, fast melt tablets, and/or mini-tablets. The dosage form can be of any shape suitable for oral administration of a drug, such as spheroidal, cube-shaped oval, or ellipsoidal.

The thickness of the polymer in the formulations, the amounts and types of polymers, and the ratio of water-soluble polymers to water-insoluble polymers in the modified-release formulations are generally selected to achieve a desired release profile of (R)-verapamil, a derivative thereof or a pharmaceutically acceptable salt thereof. For example, by increasing the amount of water-insoluble-polymer relative to the water-soluble polymer, the release of the drug may be delayed or slowed.

The amount of the dose administered, as well as the dose frequency, will vary depending on the particular dosage form used and route of administration. The amount and frequency of administration will also vary according to the age, body weight, and response of the individual subject. Typical dosing regimens can readily be determined by a competent physician without undue experimentation. It is also noted that the clinician or treating physician will know how and when to interrupt, adjust, or terminate therapy in conjunction with individual subject response.

In general, the total daily dosage for treating, preventing, and/or managing the abnormal increases in gastrointestinal motility and/or the intestinal conditions that cause the same with any of the formulations according to the present invention is from about 1 mg to about 1000 mg, or about 1, 5, 10, 15, 20, 25, 30, 35, 40, 45, 50, 55, 60, 70, 80, 90, 100, 120, 140, 150, 160, 180, 200, 250, 300, 350, 400, 450, 500, 550, 600, 650, 700, 750, 800, 850, 900, 950, or 1000 mg, or any number in between, of (R)-verapamil, a derivative thereof, or a pharmaceutically acceptable salt thereof. For example, for an orally administered dosage form, the total daily dose may range from about 30 mg to about 600 mg, or from about 60 mg to about 480 mg, or from about 120 mg to about 480 mg, or from about 120 mg to about 240 mg. Accordingly, a single oral dose may be formulated to contain about 1, 5, 10, 15, 20, 25, 30, 35, 40, 45, 50, 55, 60, 70, 80, 90, 100, 120, 140, 150, 160, 180, 200, 220, 240, 250, 260, 280, 300, 320, 340, 350, 360, 380, 400, 420, 440, 450, 460, 480, 500, 520, 540, 550, 560, 580, or 600 mg, or any number in between, of (R)-verapamil, a derivative thereof or a pharmaceutically acceptable salt thereof. The pharmaceutical compositions containing (R)-verapamil, a derivative thereof or a pharmaceutically acceptable salt thereof may be administered in single or divided doses 1, 2, 3, 4, or more times each day. Alternatively, the dose may be delivered once every 2, 3, 4, 5, or more days. In one embodiment, the pharmaceutical compositions are administered once per day.

Any of the pharmaceutical compositions and dosage forms described herein may further comprise at least one additional pharmaceutically active compound other than (R)-verapamil, a derivative thereof or a pharmaceutically acceptable salt thereof that may or may not have MT1 receptor activity, 5-HT_{2B} receptor activity and a calcium channel activity. Such compounds may be included to treat, prevent, and/or manage the same condition being treated, prevented, and/or managed with (R)-verapamil, a derivative thereof or a pharmaceutically acceptable salt thereof, or a different one. Those of skill in the art are familiar with examples of the techniques for incorporating additional active ingredients into compositions comprising (R)-verapamil, a derivative thereof or a pharmaceutically acceptable salt thereof. Alternatively, such additional pharmaceutical compounds may be provided in a separate formulation and co-administered to a subject with (R)-verapamil, a derivative thereof or a pharmaceutically acceptable salt thereof composition according to the present invention. Such separate formulations may be administered before, after, or simultaneously with the administration of (R)-verapamil, a derivative thereof or a pharmaceutically acceptable salt thereof compositions of the present invention.

The invention is further illustrated by reference to the following examples. It will be apparent to those skilled in the art that many modifications, both to the materials and methods, may be practiced without departing from the purpose and scope of the invention.

### EXAMPLES

### Example 1

### Binding Affinity

(S)- and (R)-verapamil were obtained from AMSA (Aonima Materie Sintetiche E Affini) S.P.A. (Como, Italy) and a sister company Cosma S.P.A. (Milan, Italy). Binding activity was evaluated to assess selectivity of verapamil and its enantiomers for various receptors and receptor systems.

Tables 1-10 report the binding activity of the following receptors: calcium channel (dihydropyridine site, diltiazem site, and verapamil site), 5-HT_{2B}, 5-HT Transporter, 5-HT_{2A} (agonist), 5-HT_{2A}, 5-HT_{2C} (agonist), 5-HT_{2C} receptors, Melatonin (ML1), and Melatonin subtype MT-1 and MT-2. Each of those receptors was analyzed by methods known in the art. Initial assessment of binding at 100×10⁻⁷ M (10 micro molar) was performed in duplicate. If a compound analyzed displayed more than 30%-50% binding at that concentration, then IC₅₀ determinations (e.g., at 8 concentrations in duplicate) were made. If less than 30-50% binding was observed at 100×10⁻⁷ M, then an IC₅₀ value of >100×10⁻⁷ M was reported (along with the actual percentage of binding).

The binding activity of the reported receptors was analyzed using the following methods. The calcium channel binding affinity was examined with rat cerebral cortex cells by the method disclosed in Reyonds I. J., et al., 237 Pharmacology Exp. Theory 731-38 (1986). For verapamil and its enantiomers, binding activity was determined by the method provided in Lee H. R. et al. (1994) Life Sci., 35: 721-732. For diltiazem, felodipine, nicardipine, nimodipine and isradipine, the method disclosed in Schloemaker H. and Langer S. Z. (1985), Eur J. Pharmacol. 111: 273-277 was used.

There are three distinct binding sites on the L-type calcium channel receptor. Further, the binding affinity at a particular site can be allosterically modulated by binding at one of the other sites. Thus, testing included the affinity of the different types of calcium channel blockers at their particular site of interaction.

For the 5-HT_{2B} binding affinity with both R-verapamil and S-verapamil, human recombinant-CHO cells were used with mesulergine, as the control, and the method of Kursar, J. D., et al., 46 J. Molecular Pharmacology 227 (1994). For 5HT_{2B} binding affinity with racemic verapamil, diltiazem, felodipine, isradipine, nicardipine and nimodipine, human recombinant-CHO cells were used with 5-HT, as the control, and the method of Bonhaus D W et al., Br. J. Pharmacol. 115, 622. The 5-HT Transporter was evaluated with human recombinant-CHO cells with imipramine, as a control, and the method of Tatsumi, M., et al., 368 Eur. J. Pharmacology 277-83 (1999). The 5-HT_{2A} (agonist) affinity was examined with human recombinant-HEK 293 cells and 1-[2,5-Dimethoxy-4-iodophenyl]-2-aminopropane (DOI), as the control. The method of Bryant Hu, et al., 15 Life Science 1259-68 (1996) was used to assess the 5-HT_{2A} (agonist) binding activity. For the 5-HT_{2A} affinity characterization, human recombinant-HEK 293 cells were used along with ketanserin, as a control, with the method of Bohaus D. W., et al., 115 J. Pharmacology 622-28 (1995). For the 5-HT_{2C} (agonist) binding evaluation, human recombinant-CHO cells were used in conjunction with DOI, as a control, with the method by Bryant Hu, et al., 15 Life Science 1259-68 (1996). The 5-HT_{2C} binding affinity was examined using human recombinant cells in conjunction with RS-10221, as a control, with the method by Stam N. J., et al., 269 Eur. J. Pharmacology 339-48 (1994).

For ML1 binding affinity with racemic verapamil, diltiazem, felodipine, isradipine, nicardipine and nimodipine, chicken brain source cells were used with 125-iodomelatonin, as a control, and the method of Rivkees S A et al. (1989) Endocrinology 125: 363-368.

For melatonin MT-1 (ML1a) and MT-2 (ML1b) binding affinity with the enantiomers of verapamil, human recombinant (CHO cells) were used with 2-iodomelatonin, as control, and the methods of Witt-Enderby P A and Dubocovitch M L (1996) Mol. Pharm. 50: 166-174 and Beresford I. J. M et al. (1998) Pharmacol Exp Ther., 285: 1239-1245.

Table 1 summarizes the activity of racemic verapamil and diltiazem, felodipine, isradipine, nicardipine, and nimodipine using the above referenced methods on the relevant L-type calcium channel binding (CCB) site, the agonist 5-HT_{2B} receptor and the melatonin ML-1. Based on the data from Table 1, Table 2 presents the relative potency of the 5-HT_{2B} to CCB and ML-1 to CCB.

TABLE 1
A summary of the binding affinity on L-type
calcium channel binding (CCB) site, the agonist
5-HT_{2B} receptor and the melatonin ML-1.

| | IC₅₀ Binding (×10⁻⁷ M) | | |
|---|---|---|---|
| Compound | CCB | 5-HT_{2B} | ML-1 |
| Racemic Verapamil | 0.62 | 1.79 | 91 |
| Diltiazem | 0.17 | >100(20%) | >100(9%) |
| Felodipine | 0.031 | 13 | 58 |
| Isradipine | 0.0046 | >100(3%) | 20 |
| Nicardipine | 0.033 | 22 | 30 |
| Nimodipine | 0.025 | >100(0%) | 14 |

TABLE 2
Relative potencies of the compounds
from Table 1 compared to CCB.

| Compound | (IC₅₀: 5-HT_{2B})/(IC₅₀: CCB) | (IC₅₀: ML-1)/(IC₅₀: CCB) |
|---|---|---|
| Racemic | 1.8 | 147 |
| Verapamil | | |
| Diltiazem | >588 | >588 |
| Felodipine | 419 | 1,871 |
| Isradipine | 21,739 | 4,348 |
| Nicardipine | 667 | 909 |
| Nimodipine | >4,000 | 560 |

For each compound, a relative calcium channel binding (CCB) activity was determined by dividing the IC₅₀ observed for each compound at the 5-HT_{2B} and ML-1 receptors, respectively, by the IC₅₀ observed for the calcium channel binding activity. A calcium channel selectivity greater than 1.0 index indicates that the compound is more selective for the CCB than for the 5-HT_{2B} and/or ML-1 receptors. The higher the index number, the greater the CCB selectivity. A CCB selectivity below 1.0 indicates that the compound is more selective for the 5-HT_{2B} and/or ML-1 receptors than CCB.

The compounds described in Tables 1 and 2 are established L-type calcium channel blockers. It is evident that of those compounds, the one with the closest matching potency on 5-HT2_{B} receptors and the ML-1 receptor to CCB is racemic verapamil, since the relative selectivity index is the closest to 1.0. Because the ML-1 assay used above was based on chicken sourced tissue and ML-1 is now recognized as non-selective for the 2 sub-type receptors (MT1 and MT-2), the enantiomers of verapamil were further evaluated with respect to the MT-1 sub-type receptor as well as the 5-HT_{2B} and CCB receptors (Table 3) and their relative potencies calculated (Table 4).

TABLE 3
Evaluation of (R)- and (S)-verapamil on the L-type
calcium channel binding site, the agonist 5-HT_{2B}
receptor and the melatonin MT-1 sub-receptor.

| | IC₅₀ Binding (×10⁻⁷ M) | | |
|---|---|---|---|
| Compound | CCB | 5-HT_{2B} | MT1 |
| R-verapamil | 2.4 | 1.1 | 0.55 |
| S-verapamil | 0.72 | 0.87 | 22 |

TABLE 4
Relative potencies of (R)- and (S)-verapamil compared to CCB.

| Compound | (IC₅₀: 5-HT_{2B})/(IC₅₀: CCB) | (IC₅₀: MT1)/(IC₅₀: CCB) |
|---|---|---|
| R-verapamil | 0.46 | 0.23 |
| S-verapamil | 1.21 | 30.6 |

As provided in Table 2, the relative calcium channel binding selectivity was determined by dividing the IC₅₀ values of the 5-HT_{2B} and MT1 receptors, respectively, for each compound by the IC₅₀ value of the CCB. Based on Table 4, (R)-verapamil exhibited an affinity for 5-HT_{2B} and MT1 receptors greater than that for the L-type CC, whereas the (S)-enantiomer showed more affinity for CCB versus 5-HT_{2B} and MT1, as the relative potency was great than 1.0.

Since (R)-verapamil exhibited an affinity for 5-HT_{2B} and MT1 receptors, MT2, the additional ML-1 sub-receptor, was evaluated to determine whether it is selective for this sub-receptor. (R)-verapamil binding at MT2 receptors showed 0% binding at 100×10⁻⁷M (Table 5).

TABLE 5
Evaluation of (R)-verpamil on the MT sub-receptors.

IC₅₀ Binding (×10⁻⁷ M)

| Compound | MT1 | MT2 |
|---|---|---|
| R-verapamil | 0.55 | >100 (0%) |

From the data in Table 5, (R)-verapamil is highly selective for the MT1 sub-receptor and not the MT2 sub-receptor.

Table 6 further summarizes the affinity of (R)-verapamil for a series of 5-HT receptors and the relative affinity compared with 5-HT_{2B}.

TABLE 6
Evaluation of (R)-verapamil on a series of 5-HT receptors
and relative potencies compared to 5-HT_{2B}.

| Receptor (x) | IC₅₀ (×10⁻⁷M) | (IC₅₀: 5-HTₓ)/(IC₅₀: 5-HT_{2B}) |
|---|---|---|
| 2B | 1.1 | 1 (control |
| 2C | 6.6 | 6 |
| 1A | 8.2 | 7.5 |
| 2C (agonist) | 11 | 10 |
| Transporter | 16 | 14.5 |
| 2A (agonist) | 19 | 17.3 |
| 2A | 21 | 19.1 |
| 7 | 35 | 31.8 |
| 1B | >100(11%) | >91 |
| 1D | >100(17%) | >91 |
| 4E | >100(24%) | >91 |
| 5A | >100(19%) | >91 |
| 6 | >100(47%) | >91 |
| 3 | 3,400 | 3,091 |

The IC₅₀ values and the relative 5-HT_{2B} activity (i.e., selectivity index) was determine by dividing the IC₅₀ observed for each 5HT receptor by the IC₅₀ observed for the 5-HT_{2B} receptor. A value greater than 1.0 indicates that (R)-verapamil was more selective for the 5-HT_{2B} receptor than for the receptor compared with it. The higher the index number, the greater 5-HT_{2B} selectivity. A selectivity index below 1.0 indicates that the compound is more selective for the receptor being compared with the 5-HT_{2B} than the 5-HT_{2B} receptor. From the selectivity index values in Table 6, (R)-verapamil exhibited a selectivity for 5-HT_{2B} receptors compared with all other 5-HT receptors tested.

The affinity of (S)-verapamil and racemic verapamil for selected 5-HT receptors and the relative affinity compared with 5-HT_{2B} is summarized in Tables 7 and 8 respectively.

TABLE 7
Evaluation of (S)-verapamil on a series of 5-HT receptors
and relative potencies compared to 5-HT_{2B}.

| Receptor (x) | IC₅₀ (×10⁻⁷M) | (IC₅₀: 5-HTₓ)/(IC₅₀: 5-HT_{2B}) |
|---|---|---|
| 2B | 0.87 | 1 (control) |
| 2a(agonist) | 1.0 | 1.1 |
| 2c(agonist) | 2.3 | 2.6 |
| 2a | 2.4 | 2.8 |
| Transporter | 5.9 | 6.8 |
| 2c | 6.6 | 7.6 |

TABLE 8
Evaluation of racemic verapamil on a series of 5-HT
receptors and relative potencies compared to 5-HT_{2B}.

| Receptor (x) | IC₅₀ (×10⁻⁷M) | (IC₅₀: 5-HTₓ)/(IC₅₀: 5-HT_{2B}) |
|---|---|---|
| 2B | 1.79 | 1 (control) |
| 2a (agonist) | 2.81 | 1.6 |
| 2c (agonist) | 4.11 | 2.3 |
| 2a | 7.33 | 4.1 |
| Transporter | 1.07 | 0.6 |
| 2c | 8.12 | 4.5 |

From the selectivity index values presented in Tables 7 and 8, unlike (R)-verapamil, both the S-isomer and racemic verapamil are not selective for 5-HT_{2B}, as (S)-verpamil and racmic verapamil exhibited selectivity values below 1.0 or around 1.0.

To determine whether (R)-verapamil is selective for other receptor systems, the IC₅₀ values of other receptor systems were determined. Those results are summarized at Table 9.

TABLE 9
Evaluation of (R)-verapamil on other receptor systems.

| Receptor | IC₅₀ (×10⁻⁷ M) |
|---|---|
| Sigma2 | 8.4 |
| Sigma1 | 9.1 |
| Alpha adrenergic 1A | 13 |
| Na channel | 18 |
| Alpha adrenergic 2A | 19 |
| Dopamine D2L | 27 |
| Alpha adrenergic 1B | 31 |
| Dopamine D3 | 32 |
| Alpha adrenergic 1D | 82 |
| Sst1 | 75 |

As provided in Table 3, (R)-verapamil exhibited IC₅₀ values for CCB, 5-HT_{2B} and MT1 respectively, of 2.4, 1.1, and 0.55. Those values compared with the IC₅₀ values presented in Table 9 demonstrates that (R)-verapamil does not exhibit an affinity for those other receptor systems examined, at the concentration active for CCB, 5-HT_{2B}, and MT-1.

From all the above data, the target (R)-verapamil (i.e., free, unbound) concentration range is from about 0.1 to about 3×10⁻⁷M, and the profile of receptor binding affinity is as described in Table 10.

TABLE 10
Summarized receptor binding affinity for (R)-verapamil.

% Binding

| Receptor | IC₅₀ (×10⁻⁷ M) | 0.1 | 0.3 | 1.0 | 3.0 |
|---|---|---|---|---|---|
| MT-1 | 0.6 | 13 | 35 | 67 | 83 |
| 5-HT_{2B} | 1.1 | 0 | 10 | 39 | 68 |
| Ca Channel | 2.2 | 6 | 31 | 33 | 56 |

### Example 2

### Prophylaxis of Migraine

(R)-Verapamil is administered to patients who have been diagnosed as suffering from migraines including common or classic migraine, chronic cluster headache and mixed headache. (R)-Verapamil is administered in the form of oral tablets at daily doses of about 60 mg to about 320 mg/day over a period of 26 weeks. Weekly headache scores are recorded. Therapeutic benefit is demonstrated in reduced migraine and headache frequency, duration, and intensity. Safety is assessed including monitoring effects on blood pressure and heat rate and shows minimal adverse effects and good tolerability.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method comprising administering a composition comprising a therapeutically effective amount of (R)-verapamil, a derivative thereof, or a pharmaceutically acceptable salt thereof, wherein the composition treats at least one condition having MT1 receptor, 5-HT_{2B} receptor, and L-type calcium channel activity in a subject in need thereof and releases the (R)-verapamil, a derivative thereof or a pharmaceutically acceptable salt thereof to exhibit a co-primary activity on the MT1 receptor, the 5-HT_{2B} receptor, and the L-type calcium channel.

2. The method according to claim 1, wherein the composition releases the (R)-verapamil, a derivative thereof or a pharmaceutically acceptable salt thereof to exhibit at least five times more activity on the 5-HT_{2B} receptor compared with any other 5-HT receptor.

3. The method according to claim 1, wherein the composition releases the (R)-verapamil, a derivative thereof or a pharmaceutically acceptable salt thereof to exhibit at least five times more activity on the MT1 receptor compared with the MT2 receptor.

4. The method according to claim 1, wherein the composition releases the (R)-verapamil, a derivative thereof or a pharmaceutically acceptable salt thereof to exhibit a binding activity on the L-type calcium channel and at least equi-active binding activity on the 5-HT_{2B} and MT1 receptors.

5. The method according to claim 1, wherein the composition releases the (R)-verapamil, a derivative thereof or a pharmaceutically acceptable salt thereof to exhibit a ratio of calcium channel:5-HT_{2B}:MT1 binding activity of 1:at least 1:at least 1.

6. The method according to claim 1, wherein the (R)-verapamil, a derivative thereof, or a pharmaceutically acceptable salt thereof, is present in the composition an amount ranging from about 1 mg to about 600 mg.

7. The method according to claim 6, wherein the (R)-verapamil, a derivative thereof, or a pharmaceutically acceptable salt thereof, is administered orally and in an amount ranging from about 30 mg to 600 mg per day.

8. The method according to claim 7, wherein the (R)-verapamil, a derivative thereof, or a pharmaceutically acceptable salt thereof, is administered orally and in an amount from about 60 mg to about 480 mg per day.

9. The method according to claim 1, wherein the concentration range provided by the (R)-verapamil, a derivative thereof, or a pharmaceutically acceptable salt thereof, is from about 0.1 to about 3×10⁻⁷ M.

10. The method according to claim 1, wherein the at least one condition is chosen from non GI-motility linked or secretory linked gastrointestinal conditions, migraine headaches, cluster headaches, increased intraocular pressure, cyclic vomiting, primary pulmonary hypertension, restenosis, asthma, chronic obstructive pulmonary disease, prostatic hyperplasia, generalized anxiety disorder, panic disorders, obsessive compulsive disorders, alcoholism, depression, sleep disorders, anorexia nervosa, and diseases and/or conditions thereof.

11. The method according to claim 10, wherein the non GI-motility linked or secretory linked gastrointestinal conditions are chosen from dyspepsia, functional dyspepsia, gastro-esophageal reflux disease, and diseases and/or conditions thereof.

12. The method according to claim 10, wherein the increased intraocular pressure comprises glaucoma.

13. The method according to claim 1, wherein the at least one condition is chosen from diarrhea-related or linked symptoms, chronic diarrhea, cancer-related diarrhea, carcinoid syndrome, chemotherapy and radiotherapy linked diarrhea, AIDS related diarrhea, food intolerance and malabsorption related diarrhea, medicine linked diarrhea, celiac disease, and endocrine diseases.

14. The method according to claim 1, wherein the composition is a formulation for oral, nasal, rectal, intravaginal, parenteral, buccal, sublingual, or topical administration.

15. The method according to claim 1, wherein the composition is in a form chosen from a tablet, a capsule, a suppository, and an enema.

16. The method according to claim 1, further comprising at least one excipient.

17. The method according to claim 16, wherein the at least one excipient is chosen from starches, sugars, microcrystalline cellulose, diluents, granulating agents, lubricants, binders, disintegrating agents, wetting agents, emulsifiers, coloring agents, release agents, coating agents, sweetening agents, flavoring agents, perfuming agents, preservatives, antioxidants, plasticizers, gelling agents, thickeners, hardeners, setting agents, suspending agents, surfactants, humectants, carriers, stabilizers, and combinations thereof.

18. The method according to claim 1, further comprising at least one additional pharmaceutically active agent other than the (R)-verapamil, a derivative thereof or a pharmaceutically acceptable salt thereof.

19. The method according to claim 1, wherein the composition comprises a formulation chosen from a modified release, sustained release, controlled release, and any combination thereof.

20. The method according to claim 1, wherein the composition is administered one to five times per day.

21. The method according to claim 20, wherein the composition is administered once daily.

22. A method comprising administering a composition comprising a therapeutically effective amount of (R)-verapamil, a derivative thereof, or a pharmaceutically acceptable salt thereof, wherein the composition manages at least one condition having MT1 receptor, 5-HT_{2B} receptor, and L-type calcium channel activity in a subject in need thereof and releases the (R)-verapamil, a derivative thereof or a pharmaceutically acceptable salt thereof to exhibit a co-primary activity on the MT1 receptor, the 5-HT_{2B} receptor, and the L-type calcium channel.

23. The method according to claim 22, wherein the composition releases the (R)-verapamil, a derivative thereof or a pharmaceutically acceptable salt thereof to exhibit at least five times more activity on the 5-HT_{2B} receptor compared with any other 5-HT receptor.

24. The method according to claim 22, wherein the composition releases the (R)-verapamil, a derivative thereof or a pharmaceutically acceptable salt thereof to exhibit at least five times more activity on the MT1 receptor compared with the MT2 receptor.

25. The method according to claim 22, wherein the composition releases the (R)-verapamil, a derivative thereof or a pharmaceutically acceptable salt thereof to exhibit a binding activity on the L-type calcium channel and at least equi-active binding activity on the 5-HT_{2B} and MT1 receptors.

26. The method according to claim 22, wherein the composition releases the (R)-verapamil, a derivative thereof or a pharmaceutically acceptable salt thereof to exhibit a ratio of calcium channel:5-HT_{2B}:MT1 binding activity of 1:at least 1:at least 1.
